(19) United States
(12) Patent Application Publication
(10) Pub. No.: US 2016/0304908 A1
(43) Pub. Date: Oct. 20, 2016

(54) ENHANCED EFFICIENCY ETHANOL PRODUCTION AND SUGAR CONVERSION PROCESSES

(71) Applicant: MICROVI BIOTECH INC., Hayward, CA (US)

(72) Inventors: Fatemeh RAZAVI-SHIRAZI, Hayward, CA (US); Ameen(nmn) RAZAVI, Fremont, CA (US)

(21) Appl. No.: 15/103,233

(22) PCT Filed: Dec. 12, 2014
(86) PCT No.: PCT/US2014/070071

§ 371 Date: Jun. 9, 2016

Related U.S. Application Data
(60) Provisional application No. 61/915,961, filed on Dec. 13, 2013.

(51) Int. Cl. C12P 7/14

(52) U.S. Cl. CPC C12P 7/14; C12P 2203/00

(57) ABSTRACT

Ethanol is produced at high concentration by the fermentation of sugars using a biocatalyst comprising an open, porous hydrophilic polymeric structure having microorganisms for the bioconversion of sugar irreversibly retained therein in which the microorganisms have undergone phenotypic alteration to enhance tolerance to ethanol.

### STATEMENT REGARDING FEDERALLY SPONSORED RESEARCH OR DEVELOPMENT

None

### TECHNICAL FIELD

This invention pertains to continuous processes for bioconverting sugars to ethanol with enhanced overall process energy efficiency.

### BACKGROUND

The fermentation of sugars to ethanol is well known. Sugars may be obtained from any suitable biomass source, including, but not limited to, one or more of corn, wheat, sugar beets, oats, barley, sugar cane, sorghum, cassava, rice, and the like and from cellulosic biomass. Lignocellulosic biomass is typically treated to recover cellulose and hemicellulose which can then be converted to sugars. The starch, cellulose or hemicellulose is typically subjected to pretreatment which typically includes an enzymatic hydrolysis to produce sugars.

Sugars are fermented in an aqueous broth to ethanol using a suitable microorganism such as yeast. Both continuous and batch fermentation processes have been proposed although batch processes are extensively used. Ethanol is then recovered from the fermentation broth by distillation. Since water and ethanol form an azeotrope at about 95 percent ethanol, various azeotrope-breaking procedures have been deployed such as extractive distillation and molecular sieve sorption in order to provide dehydrated ethanol.

Ethanol has many uses. In recent years, ethanol has been used as a liquid fuel or fuel additive to petroleum-based fuels. By 2010, the production capacity of ethanol in the United States has exceeded 12 billion gallons per year, much of which is used as alternative fuel or fuel additive. The market place and competitive pressures require the production of ethanol at low cost to achieve economic viability. Accordingly, ethanol plants, especially those focused on producing fuel ethanol, are highly energy integrated and have capacities greater than 100 million gallons per year to take advantage of economies of scale.

One of the challenges faced by conventional ethanol plants is the need to provide sufficient water in the fermentation broth to avoid reaching high ethanol concentrations that adversely affect the microorganisms used for the fermentation prior to the substantially complete conversion of sugars. Additionally, microorganisms used for fermentation of sugars such as yeasts, are subject to sugar inhibition if the concentration of sugar becomes too high.

Considerable efforts have been expended to genetically modify the microorganisms, usually yeasts, to tolerate greater concentrations of ethanol. Tolerance of greater concentrations of ethanol provides important benefits to the ethanol producer. First, the heat energy required for distillation of ethanol from water per unit production of ethanol is reduced. Second, an increase in production capacity of the plant may be achieved. Non-modified yeasts generally succumb in fermentation broths containing about 15 percent ethanol (terminal concentration). Development of more tolerant strains through selection and genetic modifications of yeasts have enabled commercial fermentation operations to achieve higher terminal concentrations of ethanol but less than about 20 percent. By controlling the sugar concentration in the initial fermentation broth, commercial ethanol operations are typically able to convert about 92 to about 96 percent of the fermentable sugars. Some of the fermentable sugars are consumed to sustain the yeast, and some may be unconverted.

Shirazi, et al., in U.S. patent application Ser. No. 13/918,868, filed on Jun. 14, 2013, disclose biocatalysts having a high tolerance to the presence of ethanol. These biocatalysts comprise
i. a solid structure of hydrated hydrophilic polymer defining an interior structure having a plurality of interconnected major cavities having a smallest dimension of between about 5 and 100 microns and an HEV of at least about 1000 and
ii. a population of microorganisms capable of converting sugars to at least one organic product substantially irreversibly retained in the interior of the solid structure, said population of microorganisms being in a concentration of at least about 60 grams per liter based upon the volume defined by the exterior of the solid structure when fully hydrated.
   The microorganisms are believed to undergo phenotypic alterations enabling, inter alia, enhanced tolerance to ethanol and enhanced tolerance to sugar inhibition. The disclosed biocatalysts are particularly attractive for continuous processes for the bioconversion of fermentable sugars to ethanol as the biocatalyst is substantially devoid of solids generation, and, being a solid, enables separation of the biocatalyst from the fermentation broth. Additionally, the phenotypic alterations reduce the requirement of the microorganism for sugars for metabolic sustenance thereby enabling the bioconversion of as much as 99 percent of the fermentable sugars to bioproducts. Moreover, the biocatalyst has a long lifetime and competition with undesired microorganism is substantially eliminated. For ease of reference, these biocatalysts are herein referred to as ME biocatalysts.

### SUMMARY

In accordance with this invention, energy-efficient, continuous processes for producing ethanol by bioconverting sugars are provided. In these processes, the mass ratio of ethanol to water in the fermentation broth separated from the biocatalyst in the continuous fermentation process is at least about 1:3 thereby substantially reducing the heat energy required to separate ethanol (as an azeotrope with water) from water in the distillation.

In order to maintain the fermentation broth at a suitable viscosity and density for use in a bioreactor containing the biocatalyst, preferably ethanol is added to the liquid fermentation broth. By using ME biocatalyst, not only can high concentrations of ethanol be obtained from the fermentation, but also, the surprising tolerance of ME biocatalysts to ethanol allows ethanol itself to be used as a diluent in the bioreactor. Indeed, in some instances, ethanol can comprise at least about 50 or about 70 mass percent of the fermentation broth withdrawn from the bioreactor. Also, the ME biocatalyst is characterized by a high cell density per unit volume, which can enhance bioconversion activity per unit volume of bioreactor in comparison to that of free cell fermentation bioreaction systems.

In its broad aspect, the process of this invention for producing ethanol by bioconverting sugars comprise:
a. continuously supplying to a bioreactor fermentable sugar, water and optionally ethanol, wherein the mass ratio of fermentable sugar to water being supplied is between about 1:2 and about 25:1, and the mass ratio of ethanol to fermentable sugar being supplied is between about 0:1 to about 10:1, whereby a liquid fermentation broth is provided in said bioreactor;
b. contacting the fermentation broth with biocatalyst in said bioreactor under bioconversion conditions for a time sufficient to bioconvert at least about 90 mass percent of said fermentable sugar to bioproducts comprising ethanol and carbon dioxide, wherein said biocatalyst comprises:
   i. a solid structure of hydrated hydrophilic polymer defining an interior structure having a plurality of interconnected major cavities having a smallest dimension of between about 5 and about 100 microns and an HEV (Hydration Expansion Volume) of at least about 1000 and
   ii. a population of microorganisms capable of converting said fermentable sugars to ethanol and carbon dioxide, said population of microorganisms being substantially irreversibly retained in the interior of the solid structure, said population of microorganisms being in a concentration of at least about 60 grams per liter based upon the volume defined by the exterior of the solid structure when fully hydrated,
c. continuously withdrawing carbon dioxide from said bioreactor and withdrawing fermentation broth from said bioreactor, wherein said withdrawn fermentation broth has a mass ratio of ethanol to water of at least about 1:3; and
d. distilling at least a portion of the withdrawn fermentation broth to provide an ethanol-enriched product stream and an ethanol-depleted bottoms fraction.

The fermentable sugar supplied to the bioreactor can be crystalline sugar, a slurry containing solids of fermentable sugar (massecuite or undissolved sugar crystals in at least one of water and ethanol) or as a syrup or molasses. The ethanol, if used, can be admixed with the fermentable sugars prior to being passed to the bioreactor or can be introduced into the bioreactor. The ethanol being supplied for admixture with the fermentable sugars or introduction into the bioreactor is preferably at least a portion, more preferably an aliquot portion, of the fermentation broth withdrawn from the bioreactor.

### BRIEF DESCRIPTION OF THE DRAWING

FIG. 1 is a schematic depiction of an apparatus useful in the practice of processes of this invention.

### DETAILED DISCUSSION

All patents, published patent applications and articles referenced herein are hereby incorporated by reference in their entirety.

### Definitions

As used herein, the following terms have the meanings set forth below unless otherwise stated or clear from the context of their use.

The use of the terms “a” and “an” is intended to include one or more of the element described. Lists of exemplary elements are intended to include combinations of one or more of the element described. The term “may” as used herein means that the use of the element is optional and is not intended to provide any implication regarding operability.

Adhering to the solid structure of the biocatalyst means that the microorganisms are located in cavities in the interior of the biocatalyst and are substantially irreversibly retained therein although extraordinary conditions and treatments (i.e., not normal bioconversion conditions for bioconversion using the microorganisms) might be able in some instances to cause the microorganism to exit the biocatalyst. Adhering includes surface attachment to the polymer forming the walls of the catalyst as well as where the retained microorganisms are proximate to a polymeric surface, e.g., within about 10 or 20 microns, but not directly contacting the surface. Adhering thus includes physical and electrostatic adherence. In some instances, the polymer used to make the biocatalyst may become embedded in the extracellular polymeric substance around a cell or even in or on the cell wall of the microorganism.

Bioconversion activity is the rate of consumption of substrate per hour per gram (wet) of microorganism. Where an increase or decrease in bioconversion activity is referenced herein, such increase or decrease is ascertained under similar bioconversion conditions including concentration of substrate and product in the aqueous medium. Bioconversion activity to bioproduct is the rate of production of the bioproduct per hour per gram of microorganism.

Bioconversion conditions include conditions of temperature, pressure, oxygenation, pH, and nutrients (including micronutrients) and additives required or desired for the microorganisms in the biocatalyst. Nutrients and additives include growth promoters, buffers, antibiotics, vitamins, minerals, nitrogen sources, and sulfur sources and carbon sources where not otherwise provided.

Biofilm means an aggregate of microorganisms embedded within an extracellular polymeric substance (EPS) generally composed of polysaccharides, and may contain other components such as one or more of proteins, extracellular DNA and the polymer used to make the biocatalyst. The thickness of a biofilm is determined by the size of the aggregate contained within a continuous EPS structure, but a continuous EPS structure does not include fibrils that may extend between separated biofilms. In some instances, the biofilm extends in a random, three dimensional manner, and the thickness is determined as the maximum, straight line distance between the distal ends. A thin biofilm is a biofilm which does not exceed about 10 microns in any given direction.

A state of essential stasis means that a microorganism population has undergone a substantial cessation of metabolic bioconversion activity but can be revived. The existence of an essential stasis condition can be ascertained by measuring bioconversion activity. The essential stasis condition may be aerobic, anoxic or anaerobic which may or may not be the same as that of normal operating conditions for the microorganism. Where stasis is sought, the temperature is typically in the range of about 0° C. to about 25° C., say, about 4° C. to about 15° C. which may be different from the temperatures used at normal operating conditions.

An exo-network is a community of spaced-apart microorganisms that can be in the form of individual cells or biofilms that are interconnected by extracellular polymeric substance in the form of strands. The spacing between the microorganisms or biofilms in the exo-network is sufficient to enable the passage of nutrients and substrates there between and is often at least about 0.25, say, at least about 0.5, micron and may be as large as about 5 or about 10 microns or more.

Exterior skin is an exterior layer of polymer on the biocatalyst that is less open than the major channels in the interior structure of the biocatalyst. A biocatalyst may or may not have a skin. Where a skin is present, it may or may not have surface pores. Where no surface pores are present, fluids diffuse through the skin. Where pores are present, they often have an average diameter of between about 1 and about 10 microns.

Fermentable sugars are sugars that are capable of being bioconverted by microorganisms in the biocatalyst to ethanol and carbon dioxide and may in addition produce other metabolites. For instance, if the microorganism is common yeast, C5 (five carbon) sugars are substantially not fermentable but C6 (six carbon) sugars can be fermented and thus are fermentable sugars.

Fully hydrated means that a biocatalyst is immersed in water at 25° C. until no further expansion of the superficial volume of the biocatalyst is perceived.

The “Hydration Expansion Volume” (HEV) for a biocatalyst is determined by hydrating the biocatalyst in water at about 25° C. until the volume of the biocatalyst has stabilized and measuring the superficial volume of the biocatalyst (V_{w}), removing the biocatalyst from water and removing excess water from the exterior, but without drying, and immersing the biocatalyst in ethanol at about 25° C. for a time sufficient that the volume of the biocatalyst has stabilized and then measuring the superficial volume of the biocatalyst (Vₛ).

The HEV in volume percent is calculated as the amount of [V_{w}/Vₛ]×100%. To assure dehydration with the ethanol, either a large volume ratio of ethanol to biocatalyst is used or successive immersions of the biocatalyst in fresh ethanol are used. The ethanol is initially dehydrated ethanol.

Irreversibly retained and substantially irreversibly retained mean that the microorganisms are adhering to polymeric structures defining open, porous cavities. Irreversibly retained microorganisms do not include microorganisms located on the exterior surface of a biocatalyst. A microorganisms is irreversibly retained even if the biocatalyst has exterior pores of sufficient size to permit egress of the microorganisms.

Highly hydrophilic polymers are polymers to which water is attracted, i.e., are hydroscopic. Often the polymers exhibit, when cast as a film, a water contact angle of less than about 60°, and sometimes less than about 45°, and in some instances less than about 10°, as measured by the sessile drop method using a 5 microliter drop of pure distilled water.

Highly hydrated means that the volume of the biocatalyst (excluding the volume of the microorganisms) is at least about 90 percent water.

A matrix is an open, porous, polymeric structure and is an article of manufacture having an interconnected plurality of channels or cavities (herein “major cavities”) defined by polymeric structures, said cavities being between about 5 and 100 microns in the smallest dimension (excluding any microorganisms contained therein), wherein fluid can enter and exit the major cavities from and to the exterior of the matrix. The porous matrix may contain larger and smaller channels or cavities than the major cavities, and may contain channels and cavities not open to the exterior of the matrix. The major cavities, that is, open, interconnected regions of between about 5 or about 10 to about 70 or about 100 microns in the smallest dimension (excluding any microorganism contained therein) have nominal major dimensions of less than about 300, preferably less than about 200, microns, and sometimes a smallest dimension of at least about 10 microns. The term open, porous thus refers to the existence of channels or cavities that are interconnected by openings there between.

Permeable means that a component can enter or exit the major cavities from or to the exterior of the biocatalyst.

A phenotypic change or alternation or phenotypic shift is a change in a microorganism's traits or characteristics from environmental factors and is thus different from a change in the genetic make-up of the microorganism.

Population of microorganisms refers to the number of microorganisms in a given volume and include substantially pure cultures and mixed cultures.

Quiescent means that the aqueous medium in a biocatalyst is still; however, flows of nutrients and substrates and bioproducts can occur through the aqueous medium via diffusion and capillary flow.

Retained solids means that solids are retained in the interior of the biocatalyst.

The solids may be retained by any suitable mechanism including, but not limited to, restrained by not being able to pass through pores in the skin of a biocatalyst, by being captured in a biofilm or a polysaccharide structure formed by microorganisms, by being retained in the polymeric structure of the biocatalyst, or by being sterically entangled within the structure of the biocatalyst or the microorganisms.

Smallest dimension means the maximum dimension of the shortest of the maximum dimensions defining the length, width and height of a major cavity. Usually a preponderance of the major cavities in a matrix are substantially width and height symmetrical. Hence the smallest dimension can be approximated by the maximum width of a cavity observed in a two dimensional cross section, e.g., by optical or electronic microscopy.

A solubilized precursor for the polymer is a monomer or prepolymer or the polymer itself that is dissolved or dispersed such that solids cannot be seen by the naked eye and is stable. For instance, a solid can be highly hydrated and be suspended in an aqueous medium even though the solid is not dissolved.

A stable population of microorganisms means that the population of microorganisms does not decrease by more than about 50 percent nor increase by more than about 400 percent.

Sugar means carbohydrates having 5 to 12 carbon atoms and includes, but is not limited to, D-glyceraldehyde, L-glyceraldehyde, D-erythrose, L-erythrose, D-threose, L-threose, D-ribose, L-ribose, D-lyxose, L-lyxose, D-allose, L-allose, D-altrose, L-altrose 2-keto-3-deoxy D-gluconate (KDG), D-mannitol, guluronate, mannuronate, mannitol, lyxose, xylitol, D-glucose, L-glucose, D-mannose, L-mannose, D-idose, L-idose, D-galactose, L-galactose, D-xylose, L-xylose, D-arabinose, L-arabinose, D-talose, L-talose, glucuronate, galacturonate, rhamnose, fructooligosaccharide (FOS), galactooligosaccharide (GOS), inulin, mannan oligosaccharide (MOS), oligoalginate, mannuronate, guluronate, alpha-keto acid, or 4-deoxy-L-erythro-hexoselulose uronate (DEHU).

The wet weight or wet mass of cells is the mass of cells from which free water has been removed, i.e., are at the point of incipient wetness. All references to mass of cells is calculated on the basis of the wet mass of the cells.

References to organic acids herein shall be deemed to include corresponding salts and esters.

References to matrix dimensions and volumes herein are of fully hydrated matrices unless otherwise stated or clear from the context.

### Process

The processes of this invention provide for the bioconversion of fermentable sugars to ethanol. The processes of this invention are broadly applicable to any source of fermentable sugars such as from any suitable biomass source, including, but not limited to, one or more of corn, wheat, sugar beets, oats, barley, sugar cane, sorghum, cassava, rice, and the like and from cellulosic biomass. Lignocellulosic biomass is typically treated to recover cellulose and hemicellulose which can then be converted to sugars. Fermentable sugar may be derived or obtained from lignocellulose. The biomass is typically subjected to pretreatment which typically includes an enzymatic hydrolysis to convert cellulose, hemicellulose and/or starches to sugars. The fermentable sugar can be supplied to the bioreactor in any suitable form including, but not limited to, solid granules, slurries containing undissolved sugar solids, molasses and syrups. Where undissolved sugar solids are introduced into the bioreactor, sufficient water is present in the bioreactor to dissolve the sugars. It is not essential that all undissolved sugars introduced into the bioreactor become dissolved in the bioreactor, and the fermentation broth withdrawn from the bioreactor can contain undissolved sugar. The sugars may be fermentable or a combination of fermentable and non-fermentable sugars. Since the ME biocatalyst substantially irreversibly retains the microorganism, more than one type ME biocatalyst, each containing different microorganisms, or an ME biocatalyst containing two or more different types of microorganisms, can be used in the same bioreactor to expand the types of sugars that can be fermented.

In accordance with this invention, the mass ratio of fermentable sugar to water being supplied to the bioreactor is between about 1:2 and about 25:1, and more preferably is in the range of about 1:1 to about 20:1. All or a portion of the water to the bioreactor may be in admixture with the fermentable sugars. Where only a portion of the water supplied to the bioreactor is in admixture with the sugars prior to introduction into the bioreactor, the remaining water may be separately introduced; however, to reduce heat load on the distillation column, no, or very little, water is added (except that present in an ethanol stream used to reduce the viscosity of the sugar feed as discussed below). The separate water supply may be derived from one or more of fresh water, evaporation condensate, recycle ethanol, and the like.

Preferably, the fermentation broth at the point of entry into the bioreactor, at the temperature of the bioreactor, has a viscosity of less than about 0.5, and more preferably less than about 0.1, and sometimes between about 0.001 and about 0.05, Pascal-seconds. The viscosity of the fermentation broth will depend upon conditions of temperature and pressure and the types and concentrations of components in the fermentation broth. As the fermentation broth will contain sugar being introduced into the bioreactor, the sugars (fermentable and non-fermentable) often are significant contributors to the viscosity. In general, the higher the mass ratio of total sugars to water in the fermentation broth, the higher the viscosity. Hence, the viscosity of the broth decreases as fermentation progresses. The sugars also affect the density of the fermentation broth with higher sugar concentrations having higher densities. As the sugars are fermented, the density of the broth decreases, and the increasing concentration of ethanol further reduces the density of the broth. Thus, the fermentation broth in the continuous bioreactor varies not only in sugar concentration but also viscosity and density.

The preferred processes of this invention supply ethanol to the bioreactor which serves to reduce viscosity and density of the fermentation broth and facilitates mass transfer within the fermentation broth and thus contact with the ME biocatalyst in the bioreactor. In some instances, on a mass basis, ethanol reduces viscosity to a greater extent than does water. As the ME biocatalysts enable the microorganisms to function in the presence of substantial ethanol concentrations including, in some instances, concentrations as high as 95 mass percent, the mass ratio of ethanol introduced into the bioreactor can be substantial. The mass ratio of ethanol introduced into the bioreactor to fermentable sugar introduced into the bioreactor can be up to about 10:1, and this mass ratio of ethanol to total sugar can be up to about 10:1. In some instances, this mass ratio of ethanol to fermentable sugars is in the range of about 0.05:1 to about 8:1. The mass ratio of ethanol introduced into the bioreactor to water introduced into the bioreactor is at least about 1:3, and preferably is at least about 1:2, say between about 1:2 to about 10:1.

The ethanol for introduction into the bioreactor may be from any convenient source. The preferred source is a recycle of the withdrawn fermentation broth from the bioreactor. The recycle stream may be an aliquot portion of the fermentation broth withdrawn from the bioreactor or may be a portion which has been treated, e.g., to remove or concentrate solids such as by centrifugation or filtration. The recycled ethanol may be admixed with the sugar-containing feed prior to being supplied to the bioreactor, or all or a portion can be directly introduced into the bioreactor. Especially where the sugar-containing feed is highly viscous, mechanical mixing of the ethanol with the sugar-containing feed may be desirable. The relative mass portions of ethanol to water in the recycle ethanol will depend upon the steady-state concentration of ethanol in the fermentation broth withdrawn from the bioreactor.

In some instances it is desired to remove water from a sugar-containing aqueous solution prior to its being supplied to the bioreactor. The concentration of the sugar-containing aqueous solution can be effected by any suitable unit operation. Typically evaporation is used. Consequently, removal of water prior to the supply of the sugar to the bioreactor is less energy intensive than removing that water from the withdrawn fermentation broth via a multistage distillation where ethanol is recovered. In some preferred aspects of the processes of this invention, a sugar-containing aqueous solution contains less than about 50, say, less than about 30, mass percent water and recycle ethanol is admixed with this aqueous solution to provide the sought sugar:water mass ratio for introduction into the bioreactor. The water removed by evaporation can be condensed and used for any suitable purpose, including, but not limited to, process water.

Where the sugar-containing feed is supplied as an aqueous solution, in some instances it is desired to remove non-fermentable solids from the aqueous solution to minimize the presence of debris in the bioreactor. Any suitable unit operation may be used including, but not limited to, filtration and centrifugation. Where ethanol is added to reduce the viscosity of the aqueous solution, it is preferably added prior to the solid-removal unit operation to facilitate solids removal.

To achieve bioconversion conditions, the bioreactor can be supplied with micronutrients and other additives for the fermentation such as buffers as are well known in the art. These micronutrients and other additives can be contained in the sugar feed or otherwise provided to the bioreactor.

The sugar is fermented in the bioreactor to provide ethanol and carbon dioxide.

Other metabolites, such as acetate, may also be generated. The bioreactor is maintained under bioconversion conditions. The optimal bioconversion conditions will depend upon the type of microorganism, or combination of types of microorganisms, used for the bioconversion of the sugars. In general, the ME biocatalysts enable microorganisms to effect the sought bioconversion over a broader range of conditions than do the same microorganisms in a free suspension. Often the average temperature of the fermentation broth in the bioreactor is within the range of about 20° C. to about 40° C. or about 45° C., frequently between about 30° C. to about 40° C., for mesophiles. The pressure can also fall within a broad range. However, to reduce capital and operating costs, the pressure at the head of the bioreactor is usually in the range of about 50 to about 1000, say, about 90 to about 200, kPa absolute. The pH of the fermentation broth is often in the range of about 4 to about 8, say, about 4.5 to about 6.5.

Continuous processes are preferred especially since the ME biocatalysts can provide high concentrations of microorganisms per unit volume of bioreactor, and thus, together with the enhanced bioconversion rate, provide for high conversion efficiencies of substrate with relatively brief average residence times in the bioreactor. The density of microorganisms in the bioreactor is frequently at least about 35 or about 50 grams (wet) per liter (based upon the population of the microorganisms in the ME biocatalyst and packing density of the ME biocatalyst in the fermentation broth), and can be as much as about 500 or more grams per liter. Often the density of microorganisms is in the range of about 50 or about 100 to about 250 grams per liter. As can be readily appreciated, the density of microorganisms in the bioreactor is affected by the physical form of the ME biocatalyst and its packing or positioning in the bioreactor.

The residence time of the fermentation broth in the bioreactor depends upon many factors including, but not limited to, the sought conversion of the fermentable sugars, the bioconversion conditions, the density of microorganisms in the bioreactor and the design of the bioreactor including its ability to facilitate contact between the sugars and the ME biocatalyst. As stated above, the high density of microorganisms that can be obtained in a bioreactor in combination with the high bioactivity of the ME biocatalysts can enable short hydraulic residence times to be obtained while achieving high bioconversions of the fermentable sugars, e.g., often less than about 3 or about 4 hours, and sometimes less than about 30 minutes. Preferably the hydraulic residence time in the bioreactor is sufficient to bioconvert at least about 95, preferably at least about 99, mass percent of the fermentable sugars. Often the ethanol-containing product from the bioreactor contains less than about 1, preferably less than about 0.5, mass percent sugar. The ethanol-containing product has a mass ratio of ethanol to water of at least about 1:3, often at least about 1:2, say, about 1:2 to about 20:1, and frequently about 1:1 to about 10:1.

The bioreactor may be of any suitable type for bioconversion containing solid biocatalyst. Bioreactors include up-flow and down-flow packed bioreactors, trickle bed bioreactors, ponds, bubble column bioreactors (using generated carbon dioxide for the gas phase), stirred bioreactors, fluidized bed bioreactors, plug flow (tubular) bioreactors, and membrane bioreactors. The biocatalyst can be freely mobile in the fermentation broth or fixed, e.g., to a structure in the reactor vessel, or can itself provide a fixed structure. More than one reactor vessel or stage can be used in a bioreactor. For instance, reactor vessels may be in parallel or in sequential flow series.

As discussed above, the bioconversion of sugar and production of ethanol result in the fermentation broth becoming less viscous and less dense as the bioconversion progresses. This phenomenon enables a wide variety of bioreactor types to be suitable for conducting the processes of this invention. For instance, in a tubular reactor, the initial viscosity of the fermentation broth hinders the movement of sugar within the broth to contact an ME biocatalyst. As the sugars become depleted, the lower viscosity provides less of a hindrance and movement of sugars within the fermentation broth is increased. Hence, the rate of sugar being bioconverted over the length of the tubular reactor is modulated, which in turn, facilitates temperature control and other operations. Similarly, the generation of carbon dioxide increases the turbulence of the liquid phase, thereby enhancing mixing in the fermentation broth and contact between sugars and the ME biocatalyst.

In some instances, a bioreactor containing a plurality of vessels or stages with fluid intermixing there between are desired to effect high bioconversion of the fermentable sugars. In such instances, at least about 2, and sometimes between about 3 and about 20, stages are used. Where the bioreactor comprises a fluidized bed bioreactor, the density of the ME biocatalyst in each stage may be selected to efficiently circulate in the density of the fermentation broth in a given stage. The co-produced carbon dioxide may be discharged from each stage or all, or a portion of the carbon dioxide may be passed to a subsequent stage to promote mixing.

The ME biocatalyst substantially, irreversibly retains the microorganisms in its interior. Typically adventitious microorganisms do not adhere to the exterior of the ME biocatalyst nor do they successfully invade the interior of the ME biocatalyst. Hence, the processes of this invention can operate for extended periods of time, often at least about 1 year, and essentially exhibit no change in product distribution.

The ethanol-containing product liquor exits the bioreactor and at least a portion is distilled to obtain a purified ethanol product. The recovery of ethanol by distillation is well known to those skilled in the art. See, for instance, U.S. Pat. No. 7,297,236 B1. Conventional ethanol processes recover ethanol from the fermentation broth by distilling to obtain an azeotrope of water and ethanol as the overhead (about 95% ethanol) and a still bottom substantially devoid of ethanol. If higher ethanol concentrations are desired, the azeotrope may be broken by azeotropic distillation or the use of molecular sieves to remove water. The still bottoms contain any sugars not bioconverted and other metabolites. Unlike conventional ethanol processes, the feed to the distillation unit operation has a higher ethanol concentration and thus significant energy savings can be obtained. Moreover, the processes of this invention do not generate solids (distillers grains) since the microorganisms are substantially irreversibly retained in the ME biocatalyst. Accordingly, capital, energy and other operating costs associated with recovering and drying distillers grains are eliminated.

In a preferred aspect of the processes of this invention, a portion of the ethanol-containing product liquor is combined with the sugar-containing feed, either by mixing with the feed or by introduction into the bioreactor, to reduce the viscosity of the fermentation broth. The amount recycled will, among other things, depend upon the sought viscosity and the composition of the ethanol-containing product liquor. In general, with higher ethanol concentrations, lesser recycle volumes are required for a given viscosity reduction, all other factors remaining the same. The recycle also assists in maintaining a high ethanol concentration in the ethanol-containing product liquor. The portion recycled should not be so high that a continuous build-up of contaminants, including but not limited to, other metabolites such as acetate, does not occur in the bioreactor.

### ME Biocatalyst

A. ME Biocatalyst Overview

The biocatalysts of this invention have a polymeric structure (matrix) defining interconnected major cavities, i.e., are open, porous matrices, in which the microorganisms are metabolically retained in the interior of the matrices, that is, the microorganisms promote the adherence rather than being physically restrained by an external structure. In the biocatalysts of this invention, the microorganisms and their communities, inter alia, regulate their population. Also, in conjunction with the sensed nature of the microenvironment in the matrices, it is believed that the microorganisms establish a spatial relationship among the members of the community.

The microorganisms that are retained in the matrices have the ability to form an exo-network. The quiescent nature of the cavities facilitate forming and then maintaining any formed exo-network. A discernable exo-network is not believed essential to achieving phenotypic alterations in the microorganism population such as population modulation and metabolic shift. Where an exo-network develops, often strands of EPS interconnect proximate microorganisms and connect microorganisms to the surface and form the exo-network. In some instances, the microorganisms form thin biofilms and these thin biofilms are encompassed in the exo-network. The biocatalysts have a substantial absence of biofilms in their interiors that are larger than thin biofilms. Hence, any biofilms that may ultimately form in the biocatalysts are relatively thin, e.g., up to about 10, and preferably up to about 2 or about 5, microns in thickness, and stable in size. Thus, each thin biofilm is often only a few cells and is connected in an exo-network.

A communication among the microorganisms is believed to occur through emitting chemical agents, including, but not limited to, autoinducers, and communication includes communications for community behavior and for signaling. Often, the preparation of the biocatalysts used in the processes of this invention can result in a population of microorganisms being initially located in the interior of the biocatalyst that is substantially that which would exist at the steady-state level. At these densities of microorganisms in the biocatalysts, community communications are facilitated which are believed to commence during the formation of the biocatalysts, and phenotypic shifts occur to enable the metabolic retention and modulate the population of microorganisms.

Another phenotypic alteration occurring in the biocatalysts, which is believed to be a result of this communication, is a metabolic shift, i.e., the metabolic functions of the community towards reproduction are diminished and the sought bioconversion continues. The population of microorganisms in the biocatalyst may tend to have an old average age due to this shift in the metabolic activity. Older microorganisms also tend to provide a more robust and sustainable performance as compared to younger cells as the older cells have adapted to the operating conditions.

Additional benefits of this communication can be an increase in community-level strength or fitness exhibited by the community in warding off adventitious microorganisms and maintaining strain-type uniformity. In some instances, the microorganisms during use of the biocatalyst may undergo natural selection to cause the strain-type in the community to become heartier or provide another benefit for the survival of the community of microorganisms. In some instances, the communication among the microorganisms may permit the population of microorganisms to exhibit multicellularity or multicellular-like behaviors. Thus the population of microorganisms in a ME biocatalyst may have microorganisms adapting to different circumstances but yet working in unison for the benefit of the community.

In some instances the porous matrix may provide modulation of the substrate and nutrients to the microorganisms to effect to optimize metabolic pathways involving substrates that are available, and these pathways may or may not be the primarily used pathways where ample substrate and other nutrients are available. Accordingly, microorganisms in the biocatalysts may exhibit enhanced bioactivity for a primarily used pathway or metabolic activity that is normally repressed.

It is also believed that the microenvironments may promote genetic exchange or horizontal gene transfer. Conjugation or bacterial mating may also be facilitated, including the transfer of plasmids and chromosomal elements. Moreover, where microorganisms lyse, strands of DNA and RNA in the microenvironments are more readily accessible to be taken up by microorganisms in these microenvironments. These phenomena can enhance the functional abilities of the microorganisms.

The biocatalysts exhibit an increased tolerance to toxins. In some instances, communications among microorganisms and the exo-network may facilitate the population establishing defenses against toxins. The community response to the presence of toxins has been observed in the biocatalysts of this invention. For instance, the biocatalysts survive the addition of toxins such as ethanol and sodium hypochlorite and the original bioconversion activity is quickly recovered thus indicating the survival of essentially the entire community.

In summary, due to the microenvironments in the biocatalyst, communication among the microorganisms and the phenotypic alterations undergone by the microorganisms, the biocatalysts provide a number of process-related advantages including, but not limited to,
no solid debris being generated,
the potential for high densities of bioactive material in a bioreactor,
stable population of microorganisms and bioactivity over extended periods of time,
metabolic shift of microorganisms towards production rather than growth and carbon flow shift,
ability of microorganisms to undergo essential stasis for extended durations,
ability to quickly respond to changes in substrate rate of supply and concentration,
attenuation of diauxic growth,
enhanced control and modulation of pH and redox balances in the microenvironment of the biocatalyst,
greater tolerance to substrate, bioproduct and contaminants,
ability to bioconvert substrate at ultralow concentrations,
ability to use slower growing and less robust microorganisms and increased resistance to competitiveness,
enhanced microorganism strain purity capabilities ,
ability to be subjected to in situ antimicrobial treatment,
ability to quickly start a bioreactor since the density of microorganism required at full operation is contained in the biocatalyst,
ability to contact biocatalyst with gas phase substrate, and
ease of separation of bioproduct from biocatalyst thereby facilitating continuous operations.

If desired, the biocatalysts may be treated to enhance the formation of the exo-network, and if desired, thin biofilms, prior to use in the metabolic process. However, performance of the porous matrices is not generally dependent upon the extent of exo-network formation, and often bioconversion activities remain relatively unchanged between the time before the microorganisms have attached to the polymeric structure and the time when extensive exo-network structures have been generated.

B. Physical Description of the Porous Matrices

The biocatalysts of this invention comprise a matrix having open, porous interior structure with bioactive material irreversibly retained in at least the major cavities of the matrix.

The matrices may be a self-supporting structure or may be placed on or in a preformed structure such as a film, fiber or hollow fiber, or shaped article. The preformed structure may be constructed of any suitable material including, but not limited to, metal, ceramic, polymer, glass, wood, composite material, natural fiber, stone, and carbon. Where self-supporting, the matrices are often in the form of sheets, cylinders, plural lobal structures such as trilobal extrudates, hollow fibers, or beads which may be spherical, oblong, or free-form. The matrices, whether self-supporting or placed on or in a preformed structure, preferably have a thickness or axial dimension of less than about 5, preferably less than about 2, say, between about 0.01 to about 1, centimeters.

The porous matrices may have an isotropic or, preferably, an anisotropic structure with the exterior portion of the cross section having the densest structure. The major cavities, even if an anisotropic structure exists, may be relatively uniform in size throughout the interior of the matrix or the size of the major cavities, and their frequency, may vary over the cross-section of the biocatalyst.

The biocatalyst has major cavities, that is, open, interconnected regions of between about 5 or about 10 to about 70 or about 100 microns in the smallest dimension (excluding any microorganisms contained therein). For the purposes of ascertaining dimensions, the dimensions of the microorganisms includes any mass in the exo-network. In many instances, the major cavities have nominal major dimensions of less than about 300, preferably less than about 200, microns, and sometimes a smallest dimension of at least about 10 microns. Often the biocatalyst contains smaller channels and cavities which are in open communication with the major cavities. Frequently the smaller channels have a maximum cross-sectional diameter of between about 0.5 to about 20, e.g., about 1 to about 5 or about 10, microns. The cumulative volume of major cavities, excluding the volume occupied by microorganisms and mass associated with the microorganisms, to the volume of the biocatalyst is generally in the range of about 40 or about 50 to about 70 or about 99, volume percent. In many instances, the major cavities constitute less than about 70 percent of the volume of the fully hydrated catalyst with the remainder constituting the smaller channels and pores. The volume fraction of the biocatalyst that constitute the major cavities can be estimated from its cross-section. The cross section may be observed via any suitable microscopic technique, e.g., scanning electron microscopy and high powered optical microscopy. The total pore volume for the matrices can be estimated from the volumetric measurement of the matrices and the amount and density of polymer, and any other solids used to make the matrices.

The ME biocatalyst is characterized by having high internal surface areas, often in excess of at least about 1 and sometimes at least about 10, square meter per gram. In some instances, the volume of water that can be held by a fully hydrated biocatalyst (excluding the volume of the microorganisms) is in the range of about 90 to about 99 or more, percent. Preferably the biocatalyst exhibits a Hydration Expansion Volume (HEV) of at least about 1000, frequently at least about 5000, preferably at least about 20,000, and sometimes between about 50,000 and about 200,000, percent.

Usually the type of polymer selected and the void volume percent of the matrices are such that the matrices have adequate strength to enable handling, storage and use in a bioconversion process.

The porous matrices may or may not have an exterior skin. Preferably the matrices have an exterior skin to assist in modulating the influx and efflux of components to and from the interior channels of the porous matrix. Also, since the skin is highly hydrophilic, and additional benefit is obtained as contaminating or adventitious microorganisms have difficulties in establishing a strong biofilm on the exterior of the biocatalyst. These contaminating microorganisms are often subject to removal under even low physical forces such as by the flow of fluid around the biocatalysts. Thus, the fouling of the biocatalyst can be substantially eliminated or mitigated by washing or by fluid flows during use.

Where present, the skin typically has pores of an average diameter of between about 1 and about 10, preferably about 2 to about 7, microns in average diameter. The pores may comprise about 1 to about 30, say, about 2 to about 20, percent of the external surface area. The external skin, in addition to providing a barrier to entry of adventitious microorganisms into the interior of the biocatalyst, is preferably relatively smooth to reduce the adhesion of microorganisms to the external side of the skin through physical forces such as fluid flow and contact with other solid surfaces. Often, the skin is substantially devoid of anomalies, other than pores, greater than about 2 or about 3 microns. Where a skin is present, its thickness is usually less than about 50, say, between about 1 and about 25, microns. It should be understood that the thickness of the skin can be difficult to discern where the porous matrix has an anisotropic structure with the densest structure being at the exterior of the matrix.

A high density of microorganisms can exist at steady-state operation within the ME biocatalysts. The combination of the flow channels and the high permeability of the polymeric structure defining the channels enable viable microorganism population throughout the matrix, albeit with a plurality of unique microenvironments and nano-environments. In some instances, when the bioactive material comprises microorganisms, the cell density based upon the volume of the matrices is at least about 100 grams per liter, preferably at least about 150 or about 200, and often between about 250 and about 750, grams per liter.

Solid-Containing ME Biocatalysts

The ME biocatalysts may contain one or more particulate solids which can be used to provide a sought density of the ME biocatalyst. The solid, if desired, may be a solid sorbent. The solid may be the hydrophilic polymer forming the structure or may be a particulate, i.e., a distinct solid structure regardless of shape) contained in the solid structure. Where the solid serves as a sorbent, it may be any suitable solid sorbent for the substrate or nutrients or other chemical influencing the sought metabolic activity such as, but not limited to, co-metabolites, inducers, and promoters or for components that may be adverse to the microorganisms such as, and not in limitation, toxins, phages, bioproducts and by-products. The solid sorbent is typically an adsorbent where the sorption occurs on the surface of the sorbent.

The particulate solids are preferably nano materials having a major dimension less than about 5 microns, preferably, between about 5 nanometers to about 3 microns. Where the solid is composed of polymer, the solid structure may be essentially entirely composed of the polymer or may be a block copolymer or polymeric mixture constituting between about 5 and 90 mass percent of the solid structure (excluding water). Where the solid is a separate particulate in the biocatalyst, the biocatalyst may comprise between about 5 to 90 mass percent of the mass of the biocatalyst (excluding water and microorganisms but including both the hydrophilic polymer and the particulates). More than one solid may be used in a biocatalyst. Preferably the solid is relatively uniformly dispersed throughout the interior of the biocatalyst although the solid may have a varying distribution within the biocatalyst. Where the distribution varies, the regions with the higher concentration of solid often are found toward the surface of the biocatalyst.

Where a particulate solid is used, the sorbent comprises an organic or inorganic material having the sought sorptive capacity. Examples of solids include, without limitation, polymeric materials, especially with polar moieties, carbon (including but not limited to activated carbon), silica (including but not limited to fumed silica) , silicates, clays, molecular sieves, and the like. The molecular sieves include, but are not limited to zeolites and synthetic crystalline structures containing oxides and phosphates of one or more of silicon, aluminum, titanium, copper, cobalt, vanadium, titanium, chromium, iron, nickel, and the like. The sorptive properties may comprise one or more of physical or chemical or quasi-chemical sorption on the surface of the solid sorbent. Thus, surface area and structure may influence the sorptive properties of some solid sorbents. Frequently the solid sorbents are porous and thus provide high surface area and physical sorptive capabilities. Often the pores in the solid sorbents are in the range of about 0.3 to 2 nanometers in effective diameter.

The solids may be incorporated into the polymeric structure in any convenient manner, preferably during the preparation of the ME biocatalyst.

Enzyme-Containing ME Biocatalysts

In another aspect, the ME biocatalysts can contain, in addition to the microorganisms, one or more extracellular enzymes in the interior of the biocatalyst to cause a catalytic change to a component which may be substrate or other nutrients, or a bioproduct or by-product or co-product of the microorganisms, or may be a toxin, phage or the like. Typically extracellular enzymes bond or adhere to solid surfaces, such as the hydrophilic polymer, solid additives, cell walls and extracellular polymeric substance. Hence, the enzymes can be substantially irreversibly retained in the interior of the biocatalyst. Due to the structure of the biocatalysts of this invention, the microorganisms and the enzymes can be in close proximity and thus effective, cooperative bioconversions can be obtained. The association of the enzymes with the interior surfaces of the biocatalyst typically increases the resistance of the enzyme or enzymes to denaturation due to changes in temperature, pH, or other factors related to thermal or operational stability of the enzymes. Also, by being retained in the biocatalyst, the use of the enzyme in a bioreactor is facilitated and undesirable post-reactions can be mitigated.

Representative enzymes for carbohydrate conversions to sugars include, without limitation: one or more enzymes in the classes of endo-glucanases, exo-glucanases, and β-glucosidases; endo-1,4-β-D-xylanases; exo-1,4-β-D-xylosidases, endo-1,4-β-D-mannanases; β-mannosidases; acetyl xylan esterases; α-glucuronidases; α-L-arabinofuranosidases; α-galactosidases; laccase; manganese peroxidase; lignin peroxidase; pectin methyl esterase; pectate lyase; polygalacturonase; rhamnoglacturonan lysase; glucuronidase; ferulic acid esterase; α-glaactosidase; p-coumaric acid esterase and cellobiohydrolase (e.g., CBHI, CBHII). The enzymes include those described by Heinzelman et al. (2009) PNAS 106: 5610-5615, herein incorporated by reference in its entirety.

The enzymes may be bound to the precursor for the hydrophilic polymer of the biocatalyst prior to the formation of the biocatalyst or may be introduced during the preparation of the biocatalyst, e.g., by addition to the liquid medium for forming the biocatalyst. There are many methods that would be known to one of skill in the art for providing enzymes or fragments thereof, or nucleic acids, onto a solid support. Some examples of such methods include, e.g., electrostatic droplet generation, electrochemical means, via adsorption, via covalent binding, via cross-linking, via a chemical reaction or process. Various methods are described in Methods in Enzymology, Immobilized Enzymes and Cells, Part C. 1987. Academic Press. Edited by S. P. Colowick and N. O. Kaplan. Volume 136; Immobilization of Enzymes and Cells. 1997. Humana Press. Edited by G. F. Bickerstaff. Series: Methods in Biotechnology, Edited by J. M. Walker; DiCosimo, R., McAuliffe, J., Poulose, A. J. Bohlmann, G. 2012. Industrial use of immobilized enzymes. Chem. Soc. Rev.; and Immobilized Enzymes: Methods and Applications. Wilhelm Tischer and Frank Wedekind, Topics in Current Chemistry, Vol. 200. Page 95-126.

C. Methods for Making ME Biocatalysts

The components, including bioactive materials, used to make the ME biocatalysts and the process conditions used for the preparation of the biocatalysts are not critical to the broad aspects of this invention and may vary widely as is well understood in the art once understanding the principles described above. In any event, the components and process conditions for making the biocatalysts with the irreversibly, metabolically retained microorganisms should not adversely affect the microorganisms.

The ME biocatalysts may be prepared from a liquid medium containing the bioactive material and solubilized precursor for the hydrophilic polymer which may be one or more of a polymerizable or solidifiable component or a solid that is fusible or bondable to form the matrix. Aqueous media are most often used due to the compatibility of most microorganisms and enzymes with water. However, with bioactive materials that tolerate other liquids, such liquids can be used to make all or a portion of the liquid medium. Examples of such other liquids include, but are not limited to liquid hydrocarbons, peroxygenated liquids, liquid carboxy-containing compounds, and the like. Mixed liquid media can also be used to prepare the biocatalyst. The mixed media may comprise miscible or immiscible liquid phases. For instance, the bioactive material may be suspended in a dispersed, aqueous phase and the polymerizable or solidifiable component may be contained in a continuous solvent phase.

The liquid medium used to prepare the ME biocatalyst may contain more than one type of microorganism, especially where the microorganisms do not significantly compete for the same substrate, and may contain one or more isolated enzymes or functional additives such as polysaccharide, solid sorbent and phosphorescent materials, as described above. Preferably, the biocatalysts contain a single type of microorganism. The concentration of the microorganisms in the liquid medium used to make the biocatalysts should at least be about 60 grams per liter. As discussed above, the concentration of microorganisms should preferably approximate the sought density of microorganisms in the biocatalyst. The relative amounts of microorganism and polymeric material in forming the biocatalyst can vary widely. The growth of the population of microorganisms post formation of the biocatalyst is contemplated as well as the potential for damage to some of the population of microorganisms during the biocatalyst-forming process. Nevertheless, higher microorganism concentrations are generally preferred, e.g., at least about 100 or about 150 grams per liter, preferably at least about 200, and often between about 250 and about 750, grams per liter of the liquid medium used to make the biocatalysts.

Any suitable process may be used to solidify or polymerize the polymeric material or to adhere or fuse particles to form the open, porous polymeric matrix with microorganism irreversibly retained therein. The conditions of suitable processes should not unduly adversely affect the microorganisms. As microorganisms differ in tolerance to temperatures, pressures and the presence of other chemicals, some matrix-forming processes may be more advantageous for one type of microorganism than for another type of microorganism.

Preferably the polymeric matrix is formed from solidification of a high molecular weight material, by polymerization or by cross-linking of prepolymer in manner that a population of microorganisms is provided in the interior of the biocatalyst as it is being formed. Exemplary of processes include solution polymerization, slurry polymerization (characterized by having two or more initial phases), and solidification by cooling or removal of solvent.

The biocatalysts may be formed in situ in the liquid medium by subjecting the medium to solidification conditions (such as cooling or evaporation) or adding a component to cause a polymerization or cross-linking or agglomeration of solids to occur to form a solid structure such as a catalyst, cross-linking agent or coagulating agent. Alternatively, the liquid medium may be extruded into a solution containing a solidification agent such as a catalyst, cross-linking or coagulating agent or coated onto a substrate and then the composite subjected to conditions to form the solid biocatalyst.

Polymeric materials used to make the biocatalysts may have an organic or inorganic backbone but have sufficient hydrophilic moieties to provide a highly hydrophilic polymer which when incorporated into the matrices exhibits sufficient water sorption properties to provide the sought Hydration Expansion Volume of the biocatalyst. Polymeric materials are also intended to include high molecular weight substances such as waxes (whether or not prepared by a polymerization process), oligomers and the like so long as they form biocatalysts that remain solid under the conditions of the bioconversion process intended for their use and have sufficient hydrophilic properties that the Hydration Expansion Volume can be achieved. As stated above, it is not essential that polymeric materials become cross-linked or further polymerized in forming the polymeric matrix.

Examples of polymeric materials include homopolymers and copolymers which may or may not be cross-linked and include condensation and addition polymers that provide high hydrophilicity and enable the Hydration Expansion Volumes to be obtained. The polymer may be a homopolymer or a copolymer, say, of a hydrophilic moiety and a more hydrophobic moiety. The molecular weight and molecular weight distribution are preferably selected to provide the combination of hydrophilicity and strength as is known in the art. The polymers may be functionalized with hydrophilic moieties to enhance hydrophilicity. Examples of hydrophilic moieties include, but are not limited to hydroxyl, alkoxyl, acyl, carboxyl, amido, and oxyanions of one or more of titanium, molybdenum, phosphorus, sulfur and nitrogen such as phosphates, phosphonates, sulfates, sulfonates, and nitrates, and the hydrophilic moieties may be further substituted with hydrophilic moieties such as hydroxyalkoxides, acetylacetonate, and the like. Typically the polymers contain carbonyl and hydroxyl groups, especially at some adjacent hydrophilic moieties such as glycol moieties. In some instances, the backbone of the polymer contains ether oxygens to enhance hydrophilicity. In some instances, the atomic ratio of oxygen to carbon in the polymer is between about 0.3:1 to about 5:1.

Polymers which may find use in forming the matrices include functionalized or non-functionalized polyacrylamides, polyvinyl alcohols, polyetherketones, polyurethanes, polycarbonates, polysulfones, polysulfides, polysilicones, olefinic polymers such as polyethylene, polypropylene, polybutadiene, rubbers, and polystyrene, nylons, polythyloxazyoline, polyethylene glycol, polysaccharides such as sodium alginate, carrageenan, agar, hyaluronic acid, chondroitin sulfate, dextran, dextran sulfate, heparin, heparin sulfate, heparin sulfate, chitosan, gellan gum, xanthan gum, guar gum, water soluble cellulose derivatives and carrageenan, and proteins such as gelatin, collagen and albumin, which may be polymers, prepolymers or oligomers, and polymers and copolymers from the following monomers, oligomers and prepolymers:
monomethacrylates such as polyethylene glycol monomethacrylate, polypropylene glycol monomethacrylate, polypropylene glycol monomethacrylate, methoxydiethylene glycol methacrylate, methoxypolyethylene glycol methacrylate, methacryloyloxyethyl hydrogen phthalate, methacryloyloxyethyl hydrogen succinate, 3-chloro-2-hydroxypropyl methacrylate, stearyl methacrylate, 2-hydroxy methacrylate, and ethyl methacrylate;
monoacrylates such as 2-hydroxyethyl acrylate, 2-hydroxypropyl acrylate, isobutyl acrylate, t-butyl acrylate, isooctyl acrylate, lauryl acrylate, stearyl acrylate, isobornyl acrylate, cyclohexyl acrylate, methoxytriethylene glycol acrylate, 2-ethoxyethyl acrylate, tetrahydrofurfuryl acrylate, phenoxyethyl acrylate, nonylphenoxypolyethylene glycol acrylate, nonylphenoxypolypropylene glycol acrylate, silicon-modified acrylate, polypropylene glycol monoacrylate, phenoxyethyl acrylate, phenoxydiethylene glycol acrylate, phenoxypolyethylene glycol acrylate, methoxypolyethylene glycol acrylate, acryloyloxyethyl hydrogen succinate, and lauryl acrylate;
dimethacrylates such as 1,3-butylene glycol dimethacrylate, 1,4-butanediol dimethacrylate, ethylene glycol dimethacrylate, diethylene glycol dimethacrylate, triethylene glycol dimethacrylate, polyethylene glycol dimethacrylate, butylene glycol dimethacrylate, hexanediol dimethacrylate, neopentyl glycol dimethacrylate, polyprene glycol dimethacrylate, 2-hydroxy-1,3-dimethacryloxypropane, 2,2-bis-4-methacryloxyethoxyphenylpropane, 3,2-bis-4-methacryloxydiethoxyphenylpropane, and 2,2-bis-4-methacryloxypolyethoxyphenylpropane;
diacrylates such as ethoxylated neopentyl glycol diacrylate, polyethylene glycol diacrylate, 1,6-hexanediol diacrylate, neopentyl glycol diacrylate, tripropylene glycol diacrylate, polypropylene glycol diacrylate, 2,2-bis-4-acryloxyethoxyphenylpropane, 2-hydroxy-1-acryloxy-3-methacryloxypropane; trimethacrylates such as trimethylolpropane trimethacrylate; triacrylates such as trimethylolpropane triacrylate, pentaerythritol triacrylate, trimethylolpropane EO-added triacrylate, glycerol PO-added triacrylate, and ethoxylated trimethylolpropane triacrylate; tetraacrylates such as pentaerythritol tetraacrylate, ethoxylated pentaerythritol tetraacrylate, propoxylated pentaerythritol tetraacrylate, and ditrimethylolpropane tetraacrylate;
urethane acrylates such as urethane acrylate, urethane dimethyl acrylate, and urethane trimethyl acrylate;
amino-containing moieties such as 2-aminoethyl acrylate, 2-aminoethyl methacrylate, aminoethyl methacrylate, dimethyl aminoethyl methacrylate, monomethyl aminoethyl methacrylate, t-butylaminoethylmethacrylate, p-aminostyrene, o-aminostyrene, 2-amino-4-vinyltoluene, dimethylaminoethyl acrylate, diethylaminoethyl acrylate, piperidinoethyl ethyl acrylate, piperidinoethyl methacrylate, morpholinoethyl acrylate, morpholinoethyl methacrylate, 2-vinyl pyridine, 3-vinyl pyridine, 2-ethyl-5-vinyl pyridine, dimethylaminopropylethyl acrylate, dimethylaminopropylethyl methacrylate, 2-vinyl pyrrolidone, 3-vinyl pyrrolidone, dimethylaminoethyl vinyl ether, dimethylaminoethyl vinyl sulfide, diethylaminoethyl vinyl ether, 2-pyrrolidinoethyl acrylate, 2-pyrrolidinoethyl methacrylate,
   and other monomers such as acrylamide, acrylic acid, and dimethylacrylamide.

Not all the above listed polymers will be useful by themselves, but may be required to be functionalized or used to form a co-polymer with a highly hydrophilic polymer.

Cross linking agents, accelerators, polymerization catalysts, and other polymerization additives may be employed such as triethanolamine, triethylamine, ethanolamine, N-methyl diethanolamine, N,N-dimethyl benzylamine, dibenzyl amino, N-benzyl ethanolamine, N-isopropyl benzylamino, tetramethyl ethylenediamine, potassium persulfate, tetramethyl ethylenediamine, lysine, ornithine, histidine, arginine, N-vinyl pyrrolidinone, 2-vinyl pyridine, 1-vinyl imidazole, 9-vinyl carbazone, acrylic acid, and 2-allyl-2-methyl-1,3-cyclopentane dione. For polyvinyl alcohol polymers and copolymers, boric acid and phosphoric acid may be used in the preparation of polymeric matrices. As stated above, the amount of cross-linking agent may need to be limited to assure that the matrices retain high hydrophilicity and the ability to have a high Hydration Expansion Volume. The selection of the polymer and cross-linking agents and other additives to make porous matrices having the physical properties set forth above is within the level of the artisan in the art of water soluble and highly hydrophilic polymer synthesis.

The ME biocatalysts may be formed in the presence of other additives which may serve to enhance structural integrity or provide a beneficial activity for the microorganism such as attracting or sequestering components, providing nutrients, and the like. Additives can also be used to provide, for instance, a suitable density to be suspended in the aqueous medium rather than tending to float or sink in the broth. Typical additives include, but are not limited to, starch, glycogen, cellulose, lignin, chitin, collagen, keratin, clay, alumina, aluminosilicates, silica, aluminum phosphate, diatomaceous earth, carbon, polymer, polysaccharide and the like. These additives can be in the form of solids when the polymeric matrices are formed, and if so, are often in the range of about 0.01 to about 100 microns in major dimension.

If desired, the biocatalyst may be subjected to stress as is known in the art. Stress may be one or more of starvation, chemical or physical conditions. Chemical stresses include toxins, antimicrobial agents, and inhibitory concentrations of compounds. Physical stresses include light intensity, UV light, temperature, mechanical agitation, pressure or compression, and desiccation or osmotic pressure. The stress may produce regulated biological reactions that protect the microorganisms from shock and the stress may allow the hardier microorganisms to survive while the weaker cells die.

### Microorganisms

The ME biocatalyst comprises microorganisms, the microorganisms may be unicellular or may be multicellular that behaves as a single cell microorganism such as filamentous growth microorganisms and budding growth microorganisms. Often the cells of multicellular microorganisms have the capability to exist singularly. The microorganisms can be of any type, including, but not limited to, those microorganisms that are aerobes, anaerobes, facultative anaerobes, heterotrophs, autotrophs, photoautotrophs, photoheterotrophs, chemoautotrophs, and/or chemoheterotrophs. The cellular activity, including cell growth can be aerobic, microaerophilic, or anaerobic. The cells can be in any phase of growth, including lag (or conduction), exponential, transition, stationary, death, dormant, vegetative, sporulating, etc. The one or more microorganisms be a psychrophile (optimal growth at about −10° C. to about 25° C.), a mesophile (optimal growth at about 20-about 50° C.), a thermophile (optimal growth about 45° C. to about 80° C.), or a hyperthermophile (optimal growth at about 80° C. to about 100° C.). The one or more microorganisms can be a gram-negative or gram-positive bacterium. A bacterium can be a cocci (spherical), bacilli (rod-like), or spirilla (spiral-shaped; e.g., vibrios or comma bacteria). The microorganisms can be phenotypically and genotypically diverse.

The microorganisms can be a wild-type (naturally occurring) microorganism or a recombinant (including, but not limited to genetically engineered microorganisms) microorganism. A recombinant microorganism can comprise one or more heterologous nucleic acid sequences (e.g., genes). One or more genes can be introduced into a microorganism used in the methods, compositions, or kits described herein, e.g., by homologous recombination. One or more genes can be introduction into a microorganism with, e.g., a vector. The one or more microorganisms can comprise one or more vectors. A vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain a means for self-replication. The vector can, when introduced into a host cell, integrate into the genome of the host cell and replicate together with the one or more chromosomes into which it has been integrated. Such a vector can comprise specific sequences that can allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants. Means of genetically manipulating organisms are described, e.g., Current Protocols in Molecular Biology, last updated Jul. 25, 2011, Wiley, Print ISSN: 1934-3639. In some embodiments, one or more genes involved in byproduct formation are deleted in a microorganism. In some embodiments, one or more genes involved in byproduct formation are not deleted. Nucleic acid introduced into a microorganism can be codon-optimized for the microorganism. A gene can be modified (e.g., mutated) to increase the activity of the resulting gene product (e.g., enzyme). Sought properties in wild-type or genetically modified microorganisms can often be enhanced through a natural modification process, or self-engineering process, involving multigenerational selective harvesting to obtain strain improvements such as microorganisms that exhibit enhanced properties such as robustness in an environment or bioactivity. See, for instance, Ben-Jacob, et al., Self-engineering capabilities of bacteria, J. R. Soc. Interface 2006, 3, doi: 10.1098/rsif.2005.0089, 22 Feb. 2006.

The selected microorganism to be used in a biocatalyst can be targeted to the sought activity. The biocatalysts thus often contain substantially pure strain types of microorganisms and, because of the targeting, enable high bioactivity to be achieved and provide a stable population of the microorganism in the biocatalyst.

Examples of microorganisms for sugar conversion include, but are not limited to, wild-type and modified microorganisms such as Saccharomyces cervisiae strains TMB 3400, TMB 3006, and 424A (LNF-ST), Pachysolen tannophilus, modified E. coli strains; and the like. See, U.S. Patent Application Publication 2010/0285552, hereby incorporated by reference. Examples of microorganisms capable of bioconverting pentose to ethanol include, but are not limited to, Zymomonas mobilis, Pichia stipitis, Pichia pastoris, Candida shehatae, and Pachysolen tannophilus, and recombinant microorganisms such as, Escherichia, Pseudomonas, Alcaligenes, Salmonella, Shigella, Burkholderia, Oligotropha, Klebsiella, Pichia, Candida, Hansenula, Saccharomyces, including but not limited to, S. bayanus, Kluyveromyces, Comamonas, Corynebacterium, Brevibacterium, Rhodococcus, Azotobacter, Citrobacter, Enterobacter, Clostridium, including but not limited to, C. ljungdahlii, Lactobacillus, Aspergillus, Zygosaccharomyces, Dunaliella, Debaryomyces, Mucor, Torula, Torulopsis, Methylobacteria, Bacillus, Rhizobium and Streptomyces as are known in the art. See, for instance, Aristidou, et al., Conversion of Renewable Resources to Biofuels and Fine Chemicals: Current Trends and Future Prospects, in Fermentation Microbiology and Biotechnology, 2011, Third Edition, pp 225 to 261.

The following examples are provided in illustration of the biocatalysts and processes for making the biocatalysts and are not in limitation. All parts and percentages of solids are by mass and of liquids and gases are by volume unless otherwise stated or is clear from the context.

In these examples, the following general procedure is used. The microorganisms for the biocatalyst are grown under suitable planktonic conditions in an aqueous medium for the microorganisms including the presence of nutrients and micronutrients. This medium is referred to herein as the “Culture Medium”. The microorganisms used are as available and thus may be either substantially pure strains or mixed cultures. The cell density in the Culture Medium is determined by optical density. If too thick, the cell density is determined through filtration of solids and determining the mass of solids per unit volume. If the cell density of the Culture Medium is below that sought to make the biocatalyst, the Culture Medium is centrifuged or filtered to provide a denser, cell-containing fraction. A separately prepared aqueous solution of solubilized precursor is made (referred to herein as the “Polymer Solution”). Any solid additive for the biocatalysts is added to the Polymer Solution in amounts that will provide the sought amount in the biocatalyst. The Polymer Solution is mixed with a mechanical stirrer to assure uniform dispersion of the components in the aqueous medium. Where necessary to solubilize the precursor, the Polymer Solution can be heated as appropriate. In some instances, a micronutrient solution is also added to the Polymer Solution.

Aliquots of each of the Culture Medium (or dense phase from centrifugation) and Polymer Solution are admixed under mechanical stirring at about 30° C. to for a Precursor Solution. Where the microorganism is anaerobic, the Culture Medium and the mixing of the Culture Medium and Polymer Solution and all subsequent steps are maintained under anaerobic conditions by purging with nitrogen.

The Precursor Solution is then extruded through a perforated plate having orifices of about 0.75 millimeter in diameter to form droplets of about 3 millimeters in diameter. The droplets fall into a gently stirred coagulating bath of an aqueous boric acid solution having a pH of about 5. The biocatalyst is recovered from the coagulating bath and washed with distilled water. The biocatalyst, after washing, is placed in a liquid medium containing micronutrients and the substrate under suitable metabolic conditions for the microorganisms.

Table I summarizes the examples. Table II sets forth the microorganisms used in the examples. Table III sets forth the hydrophilic polymer(s) that is used in the examples. Table IV sets forth the solid additive packages used in the examples.

TABLE I

| | | Volume parts | | | Volume parts | | |
|---|---|---|---|---|---|---|---|
| | | Polymer | | Micro- | Microorganism | | Mass parts of |
| | | Solution per | | organism | culture per 100 | | Solid Additive |
| | | 100 parts of | | culture | parts of | Solid | package per liter |
| | Polymer | Precursor | Micro- | density wet | Precursor | Additive | of Precursor |
| Example | Solution | Solution | organism | weight g/L | Solution | Package | Solution |
| 1 | A | 80 | M-1 | 620 | 20 | S-1 | 1 |
| 2 | R | 45 | M-1 | 80 | 55 | S-8 | 10 |
| 3 | Q | 88 | M-2 | 650 | 12 | S-11 | 0.1 |
| 4 | M | 78 | M-2 | 675 | 22 | N/A | N/A |
| 5 | E | 70 | M-4 | 310 | 30 | N/A | N/A |
| 6 | P | 85 | M-1 | 670 | 15 | S-13 | 1.0 |
| 7 | L | 71 | M-3 | 420 | 29 | S-15 | 1.0 |
| 8 | A | 82 | M-2 | 625 | 18 | S-12 | 1.0 |
| 9 | Q | 88 | M-3 | 750 | 12 | S-6 | 1.0 |
| 10 | A | 79 | M-1 | 555 | 21 | S-3 | 0.17 |
| 11 | C | 72 | M-2 | 595 | 28 | S-7 | 1.0 |
| 12 | B | 69 | M-2 | 540 | 31 | S-2 | 1.1 |
| 13 | B | 70 | M-1 | 320 | 30 | S-5 | 0.14 |
| 14 | D | 66 | M-4 | 395 | 34 | S-9 | 3.2 |
| 15 | G | 79 | M-1 | 580 | 21 | S-16 | 0.5 |
| 16 | A | 80 | M-3 | 770 | 20 | S-2 | 0.14 |
| 17 | N | 77 | M-2 | 700 | 23 | S-12 | 1.0 |
| 18 | B | 67 | M-1 | 410 | 33 | S-14 | 0.1 |
| 19 | I | 80 | M-4 | 810 | 20 | N/A | N/A |
| 20 | J | 90 | M-1 | 940 | 10 | S-4 | 0.95 |
| 21 | S | 46 | M-4 | 90 | 54 | N/A | N/A |
| 22 | A | 82 | M-1 | 705 | 18 | S-10 | 5 |
| 23 | K | 65 | M-1 | 470 | 35 | S-17 | 0.1 |
| 24 | I | 77 | M-2 | 660 | 23 | N/A | N/A |
| 25 | L | 70 | M-4 | 555 | 30 | S-4 | 0.29 |

TABLE II

| Microorganism | |
|---|---|
| Identifier | Microorganism |
| M-1 | Saccharomyces cerevisiae Sigma ® YSC2 ™ |
| M-2 | Zymomonas mobilis ZM4 ATCC ® 31821 ™ |
| M-3 | Saccharomyces cerevisiae, Fermentis Ethanol Red ® |
| M-4 | Saccharomyces cerevisiae ATCC ® 9763 ™ |

TABLE III

| Polymer | |
|---|---|
| Solution | |
| Identifier | Composition |
| A | 8.0 wt. percent of polyvinyl alcohol available as Elvanol ® 70-04 from E. I. duPont de Nemours having a degree of |
| | hydrolysis of 98.0-98.8 mol percent; 2.0 wt. percent of sodium alginate available as Nalgin ™ MV-120 from |
| | Ingredient Solutions, Inc.; 0.5 wt. percent of medium molecular weight Poly(D-glucosamine) available as Sigma- |
| | Aldrich 448877 |
| B | 25 wt. percent of Poly(acrylamide-co-acrylic acid) potassium salt-cross-linked available as Sigma-Aldrich 432776; |
| | 0.2 wt. percent of Poly(2-hydroxyethyl methacrylate) available as Sigma-Aldrich P3932 |
| C | 14 wt. percent of poly(vinyl alcohol-co-ethylene) available as Sigma-Aldrich 414093 having an ethylene |
| | composition of 32 mol percent; 2.0 wt. percent of polyethylene glycol with an average molecular weight of 200 |
| | available as Sigma-Aldrich P3015 |
| D | 23.0 wt. percent of polyvinyl alcohol available as Elvanol ® 70-03 from E. I. duPont de Nemours having a degree |
| | of hydrolysis of 98-98.8 mol percent; 1.0% wt. percent of anhydrous calcium chloride available as Sigma-Aldrich |
| | C1016; 0.9 wt. percent of sodium alginate available as Nalgin ™ MV-120 from Ingredient Solutions, Inc. |
| E | 15.0 wt. percent of polyvinyl alcohol available as Mowial ® 28-99 from Kuraray Co., Ltd. ™ having a degree of |
| | hydrolysis of 99.0-99.8 mol percent and a molecular weight of 145,000; 3.5 wt. percent of sodium alginate |
| | available as Nalgin ™ MV-120 from Ingredient Solutions, Inc. |
| F | 21.7 wt. percent of polyethylene oxide available as POLYOX ™ WSR N-80 from The Dow Chemical Company |
| | having an approximate molecular weight of 200,000; 1.0 wt. percent of medium molecular weight Poly(D- |
| | glucosamine) available as Sigma-Aldrich 448877; 0.5 wt. percent of sodium alginate available as Nalgin ™ MV- |
| | 120 from Ingredient Solutions, Inc. |
| G | 12.0 wt. percent of Poly(acrylamide-co-acrylic acid) potassium salt-cross-linked available as Sigma-Aldrich |
| | 432776; 0.2 wt. percent of ethylene glycol dimethacrylate available as Sigma-Aldrich 335681 |
| H | 50.0 wt. percent of polyvinyl alcohol available as Elvanol ® 70-03 from E. I. duPont de Nemours having a degree |
| | of hydrolysis of 98-98.8 mol percent; 0.2 wt. percent polyaniline available as Sigma-Aldrich 577073 |
| I | 10.0 wt. percent of poly(acrylic acid) available as Sigma-Aldrich 192023 having an average molecular weight of |
| | 2000; 1.0 wt. percent of polyethylene glycol with an average molecular weight of 200 available as Sigma-Aldrich |
| | P3015 |
| J | 3.7 wt. percent of polyethylene oxide available as POLYOX ™ WSR N-80 from The Dow Chemical Company |
| | having an approximate molecular weight of 200,000; 0.5 wt. percent of anhydrous calcium chloride available as |
| | Sigma-Aldrich C1016; 0.2 wt. percent κ-Carrageenan available as Sigma-Alrdich ® 22048 |
| K | 18.1 wt. percent of poly(vinyl alcohol-co-ethylene) available as Sigma-Aldrich 414093 having an ethylene |
| | composition of 32 mol percent; 5.5 wt. percent of Poly(2-hydroxyethyl methacrylate) available as Sigma-Aldrich |
| | P3932; 1.0 wt. percent of anhydrous calcium chloride available as Sigma-Aldrich C1016 |
| L | 19.0 wt. percent of polyethylene-alt-maleic anhydride available as Sigma-Aldrich 188050 having an average |
| | molecular weight 100,000-500,000; 0.05 wt. percent of medium molecular weight Poly(D-glucosamine) available |
| | as Sigma-Aldrich 448877; 0.03 wt. percent of xantham gum from Xanthamonas campestris available as Sigma- |
| | Aldrich G1253 |
| M | 9.0 wt. percent of poly(N-isopropylacrylamide) available as Sigma-Aldrich 535311 having a molecular weight of |
| | 19,000-30,000; 2.0 wt. percent of sodium alginate available as Nalgin ™ MV-120 from Ingredient Solutions, Inc. |
| N | 4.3 wt. percent of Poly(acrylamide-co-acrylic acid) potassium salt-cross-linked available as Sigma-Aldrich 432776; |
| | 2.0 wt. percent of xantham gum from Xanthamonas campestris available as Sigma-Aldrich G1253 |
| P | 9.4 wt. percent of polyvidone available as Kollidon ® 25 Sigma-Aldrich 02286 having a degree of hydrolysis of |
| | 99+ mol percent and a molecular weight of 146,000-186,000; 2.0 wt. percent of xantham gum from Xanthamonas |
| | campestris available as Sigma-Aldrich G1253 |
| Q | 5.4 wt. percent of polyethylene oxide available as POLYOX ™ WSR N-10 from The Dow Chemical Company |
| | having an approximate molecular weight of 100,000; 2.5 wt. percent polyethylene glycol with an average |
| | molecular weight of 1450 available as Sigma-Aldrich P5402 |
| R | 33.0 wt. percent of polyethylene oxide available as POLYOX ™ WSR N-10 from The Dow Chemical Company |
| | having an approximate molecular weight of 100,000; 1.0 wt. percent of medium molecular weight Poly(D- |
| | glucosamine) available as Sigma-Aldrich 448877 |

TABLE IV

| Solid Additive | |
|---|---|
| Package Identifier | Composition |
| S-1 | Clay available as Nanomer ® I.28E from Sigma-Aldrich 682608 containing 25-30 wt % trimethyl stearyl |
| | ammonium on Montmorillonite clay base material matrix |
| S-2 | Clay available as Nanomer ® PGV hydrophilic bentonite from Sigma-Aldrich 682659 |
| S-3 | Clay available as Nanomer ® I.34MN from Sigma-Aldrich 682640 containing 25-30 wt. % methyl |
| | dihydroxyethyl hydrogenated tallow ammonium on Montmorillonite clay base material matrix |
| S-4 | Natural bentonite clay as Cloisite ® Ca++ from Southern Clay Products/Rockwood Additives |
| S-5 | Natural bentonite clay as Cloisite ® 116 from Southern Clay Products/Rockwood Additives |
| S-6 | Sodium metasilicate as granular powder available as Sigma-Aldrich 307815 |
| S-7 | Sodium hydroxidecoated silica available as Ascarite ® II from Sigma-Aldrich 223913 |
| S-8 | Starch as available from Sigma-Aldrich S4251 |
| S-9 | Starch as available from Spectrum ® M1372 |
| S-10 | Starch as available as CHARGEMASTER ® L340 from Grain Processing Corporation |
| S-11 | Aluminum oxide nanowires with diameter of 2-6 nanometers and length 200-400 nanometers as |
| | available as Sigma-Aldrich 551643 |
| S-12 | Mesostructured silica with cell window size ~15 nanometers as available as Sigma-Aldrich 560979 |
| S-13 | Chitin as available as Sigma-Aldrich C7170 |
| S-14 | Iron oxide as available as Sigma-Aldrich 310069 |
| S-15 | Fine ground silica available as MIN-U-SIL ® from U.S. Silica |
| S-16 | Polyethylene powder as MIPELON ™ from Mitsui Chemicals America, Inc. |
| S-17 | Granular activated carbon having an effective size 0.7-0.9 mm available as HYDRODARCO ® 3000 |
| | from Norit Americas |

Each of the above biocatalysts exhibit phenotypic alterations and the biocatalysts have a stable population of microorganisms and do not generate any appreciable debris from metabolic activity.

Reference is made to the drawing for purposes of facilitating the broad aspects of the processes of this invention. The drawing, however, is not in limitation of the invention. The drawing omits minor equipment such as pumps, compressors, valves, instruments and other devices the placement of which and operation thereof are well known to those practiced in chemical engineering. The drawing also all omits ancillary unit operations.

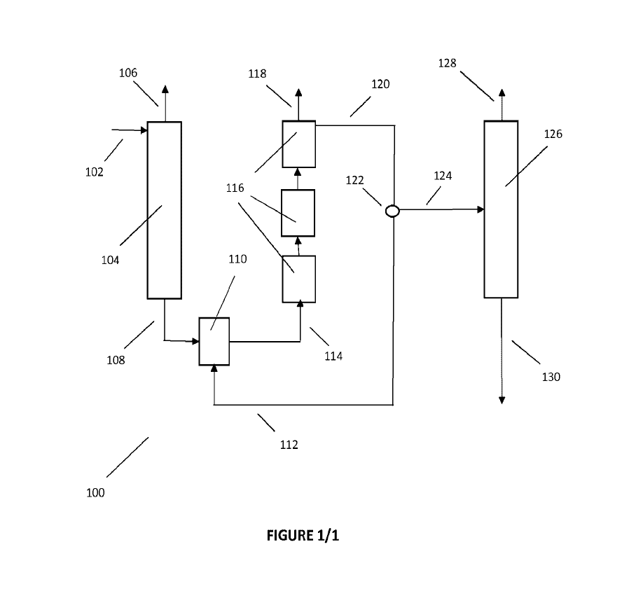

FIG. 1 schematically depicts an apparatus 100 suitable for practicing processes of this invention. As shown, a sugar-containing liquor is passed via line 102 to evaporator 104. The sugar may be from any source and is preferably devoid of solids. For purposes of discussion and not in limitation, the sugar is derived from cane sugar concentrate containing about 30 percent sugar. Evaporator 104 may be of any suitable design. As shown, evaporator 104 comprises a series of thin film, tubular evaporators externally heated by steam. Water vapor is withdrawn from evaporator 104 by line 106, and a molasses containing about 65 percent sugar exits via line 108 and is directed to mixing vessel 110. Mixing vessel 110 serves to mix the molasses with an ethanol-rich product stream from the fermentation being supplied via line 112 to provide a combined stream having a reduced viscosity, for instance, about 0.01 Pascal-seconds at a temperature of 30° C. The ethanol-rich product stream is discussed below. Preferably mixing vessel 110 contains a mechanical mixer to facilitate combination of the molasses and ethanol-rich product stream such as a screw mixer or blade mixer. The combined stream is passed from mixing vessel 110 to bioreactor 116 via line 114.

Bioreactor 116 may be of any suitable design. As shown, bioreactor 116 comprises three fluidized bed reactor stages. Bioreactor 116 contains biocatalyst, e.g., of example 5 except that clay (S-2) is added to increase the density of the biocatalyst to approximately that of the liquid phase in each stage of bioreactor 116. About 10 mass percent clay is used for the biocatalyst for the first stage, about 5 mass percent for the second stage and about 2 mass percent for the third stage. Bioreactor 116 may also contain a second biocatalyst for the bioconversion of C5 sugars, if present, to ethanol such as described in example 17. Gas phase, primarily carbon dioxide, and liquid from one stage is passed to the next bioreactor stage in the series. In the final stage, gas phase is withdrawn via line 118 and an ethanol-rich product liquor is withdrawn via line 120 and is passed to diverter valve 122. The ethanol-rich product liquor contains ethanol which was used to mix with the molasses in mixing vessel 110 and ethanol from the fermentation of sugar in bioreactor 116. The concentration of fermentable sugars in the ethanol-rich product liquor is about 0.25 grams per liter. At steady-state, the ethanol-rich product liquor contains about 50 mass percent water, 46 mass percent ethanol and less than 3 mass percent acetate.

Proportioning valve directs a portion of the ethanol-rich product liquor as the ethanol-rich product stream via line 112 to mixing vessel 110 and the remainder to distillation column 126 via line 124. The portion of the ethanol-rich product liquor passed to distillation column 126 is sufficient to prevent a build-up of liquid volume in bioreactor 116. Thus, about 50 volume percent of the ethanol-rich product liquor is passed to distillation column 126 and the balance is used as the ethanol-rich product stream for reducing the viscosity of the molasses in mixing vessel 110.

Distillation column 126 provides an ethanol product stream (95% ethanol) via line 128 and an aqueous bottoms stream substantially devoid of ethanol exits distillation column 126 via line 130.

It is claimed:

1. An energy-efficient, continuous process for producing ethanol by bioconverting sugars, the process comprising:
a. continuously supplying to a bioreactor fermentable sugar, water and optionally ethanol, wherein the mass ratio of fermentable sugar to water being supplied is between about 1:2 and about 25:1, and the mass ratio of ethanol, if used, to fermentable sugar being supplied is between about 0:1 to about 10:1, whereby a liquid fermentation broth is provided in said bioreactor;
b. contacting the fermentation broth with biocatalyst in said bioreactor under bioconversion conditions for a time sufficient to bioconvert at least about 90 mass percent of said fermentable sugar to bioproducts comprising ethanol and carbon dioxide, wherein said biocatalyst comprises:
i. a solid structure of hydrated hydrophilic polymer defining an interior structure having a plurality of interconnected major cavities having a smallest dimension of between about 5 and about 100 microns and a hydration expansion volume (HEV) of at least about 1000 and
ii. a population of microorganisms capable of converting said fermentable sugars to ethanol and carbon dioxide, said population of microorganisms being substantially irreversibly retained in the interior of the solid structure, said population of microorganisms being in a concentration of at least about 60 grams per liter based upon the volume defined by the exterior of the solid structure when fully hydrated,
c. continuously withdrawing carbon dioxide from said bioreactor and withdrawing fermentation broth from said bioreactor, wherein said withdrawn fermentation broth has a mass ratio of ethanol to water of at least about 1:3; and
d. distilling at least a portion of the withdrawn fermentation broth to provide an ethanol-enriched product stream and an ethanol-depleted bottoms fraction.

2. The process of claim 1, wherein the fermentable sugar supplied to the bioreactor is a slurry comprising solids of fermentable sugar or a syrup.

3. The process of claim 1, wherein a portion of the withdrawn fermentation broth of step (c) is recycled to the bioreactor.

4. The process of claim 3, wherein the portion of the fermentation broth being recycled to the bioreactor is admixed with fermentable sugar supplied to the bioreactor.

5. The process of claim 3, wherein the mass ratio of ethanol contained in the portion of the fermentation broth being recycled to the bioreactor is between about 1:10 to about 10:1.

6. The process of claim 1, wherein the fermentable sugar comprises hexose.

7. The process of claim 6, wherein the fermentable sugar is derived from lignocellulose.

8. The process of claim 6, wherein the fermentable sugar is cane sugar.

9. The process of claim 1, wherein the biocatalyst comprises yeast.

10. The process of claim 1, wherein hexose and pentose are supplied to the bioreactor, and hexose is the fermentable sugar.

11. The process of claim 1, wherein the fermentable sugar comprises hexose and pentose, and the bioreactor comprises a biocatalyst comprising microorganisms for bioconverting hexose to ethanol and for bioconverting pentose to ethanol.

12. The process of claim 1, wherein the withdrawn fermentation broth has a substantial absence of solids.

13. The process of claim 1, wherein the bioreactor comprises at least 2 stages or vessels.

14. The process of claim 13, wherein at least a portion of the carbon dioxide generated in one stage or vessel is passed to a subsequent stage or vessel.

15. The process of claim 13, wherein the bioreactor is a fluidized bed bioreactor.

16. The process of claim 13, wherein the bioreactor is a stirred bioreactor.

17. The process of claim 13, wherein the bioreactor is a packed bed bioreactor.
